(19) United States
(12) Patent Application Publication
(10) Pub. No.: US 2002/0160451 A1
(43) Pub. Date: Oct. 31, 2002

(54) Novel orphan receptors

(76) Inventors: Piotr J. Masiakowski, Pleasant Valley, NY (US); Jodi Morris, New City, NY (US); David M. Valenzuela, Yorktown Heights, NY (US)

Correspondence Address: Linda O. Palladino, Regeneron Pharmaceuticals, Inc., 777 Old Saw Mill River Road, Tarrytown, NY, 10591, US

(21) Appl. No.: 09/941,973

(22) Filed: Aug. 29, 2001

Related U.S. Application Data
(63) Continuation of application No. 09/128,820, filed on Aug. 4, 1998, now abandoned.

(51) Int. Cl. C07K 14/705; C12P 21/02; C12N 5/06; C07H 21/04

(52) U.S. Cl. 435/69.1; 435/325; 435/320.1; 530/350; 536/23.5

(57) ABSTRACT

The present invention provides for nucleic acid sequences that encode novel mammalian receptor polypeptides, designated HUMAN OCR10. The invention also provides assay systems that may be used to detect and/or measure ligands that bind the HUMAN OCR10 gene product. The present invention also provides for diagnostic and therapeutic methods based on the interaction between HUMAN OCR10 and agents that initiate signal transduction through binding to HUMAN OCR10.

### NOVEL ORPHAN RECEPTORS

Throughout this application various publications are referenced. The disclosures of these publications in their entireties are hereby incorporated by reference into this application.

### INTRODUCTION

The field of this invention is polypeptide molecules which regulate cell function, nucleic acid sequences encoding the polypeptides, and methods of using the nucleic acid sequences and the polypeptides. The present invention provides for novel receptor molecules, their use and assay systems useful for identifying novel ligands that interact with these preceptors.

### BACKGROUND OF THE INVENTION

The ability of ligands to bind cells and thereby elicit a phenotypic response such as development, differentiation, growth, proliferation, survival and regeneration in such cells is often mediated through transmembrane receptors. The extracellular portion of each receptor is generally the most distinctive portion of the molecule, as it provides the protein with its ligand-recognizing characteristic. In the case of receptor tyrosine kinases (RTKs), binding of a ligand to the extracellular domain results in signal transduction via an intracellular tyrosine kinase catalytic domain which transmits a biological signal to intracellular target proteins. The particular array of sequence motifs of this intracellular tyrosine kinase catalytic domain determines its access to potential kinase substrates (Mohammadi, et al., 1990, Mol. Cell. Biol. 11:5068-5078; Fantl, et al., 1992, Cell 69:413-413). For instance, growth hormone (GH) and prolactin (PRL) receptor signal transduction is mediated by a signaling system that links activation of the GH or PRL receptor at the cell surface to changes in gene transcription in the nucleus. This pathway utilizes the Jak/Stat (Janus kinase/signal transducer and activator of transcription) pathway used by many growth factors and cytokines (See Watson, et al., 1996, Rev. Reprod. 1:1-5).

The tissue distribution of a particular receptor within higher organisms provides relevant data as to the biological function of the receptor. The RTKs for some growth and differentiation factors, such as fibroblast growth factor (FGF), are widely expressed and therefore appear to play some general role in tissue growth and maintenance. Members of the Trk RTK family (Glass & Yancopoulos, 1993, Trends in Cell Biol. 3:262-268) of receptors are more generally limited to cells of the nervous system, and the neurotrophins which bind these receptors promote the differentiation of diverse groups of neurons in the brain and periphery (Lindsay, R. M, 1993, in Neurotrophic Factors, S. E. Loughlin & J. H. Fallon, eds., pp. 257-284 (San Diego, Calif., Academic Press).

The cellular environment in which a receptor is expressed may influence the biological response exhibited upon binding of a ligand to the receptor. Thus, for example, when a neuronal cell expressing a Trk receptor is exposed to a neurotrophin which binds that receptor, neuronal survival and differentiation results. When the same receptor is expressed by a fibroblast, exposure to the neurotrophin results in proliferation of the fibroblast (Glass, et al., 1991, Cell 66:405-413). Thus, it appears that the extracellular domain provides the determining factor as to the ligand specificity, and once signal transduction is initiated the cellular environment will determine the phenotypic outcome of that signal transduction.

Comparison of the rat prolactin receptor sequence with that of the mammalian growth hormone receptor sequence has demonstrated some regions of identity between the two receptors, suggesting that the receptors originate from a common ancestry and may actually belong to a larger family of receptors, all of which share certain sequence homologies and perhaps related biological function. Because ligands and their receptors appear to mediate a number of important biological functions during development (e.g., bone growth, sexual maturation) as well as in the adult (e.g., homeostasis, reproduction), the identification and isolation of novel receptors may be used as a means of identifying new ligands or to study intracellular signalling pathways that may play a crucial role during development and in the maintenance of the adult phenotype. Often such novel receptors are identified and isolated by searching for additional members of known families of receptors using, for example, PCR-based screens involving known regions of homology among receptor family members. (See, for example, Maisonpierre, et al., 1993, Oncogene 8:1631-1637). Isolation of such so called “orphan” receptors, for which no ligand is known, and subsequent determination of the tissues in which such receptors are expressed, provides insight into the regulation of the development, differentiation, growth, proliferation, survival and regeneration of cells in target tissues. Further, such receptors may be used to isolate their cognate ligands, which may then be used to regulate the development, differentiation, growth, proliferation, survival and regeneration of cells expressing the receptor.

### SUMMARY OF THE INVENTION

The present invention provides for a novel mammalian receptor, termed orphan cytokine receptor-10 (OCR10), which is highly expressed in human heart and placenta. Specifically, the present invention provides for a novel human receptor termed HUMAN OCR10. The protein appears to be related to the cytokine family of receptors which includes, but is not limited to, the interleukin-9 receptor (IL-9R), the cytokine receptor, chain, the EPO receptor, and the leptin receptor (OB-R). The present invention further provides for an isolated nucleic acid molecule encoding HUMAN OCR10.

The present invention also provides for a protein or polypeptide that comprises the extracellular domain of HUMAN OCR10 and the nucleic acid which encodes such extracellular domain.

The invention further provides for vectors comprising an isolated nucleic acid molecule encoding HUMAN OCR10 or its extracellular domain, which can be used to express HUMAN OCR10 in bacteria, yeast, insect or mammalian cells.

The present invention further provides for use of the HUMAN OCR10 receptor or its extracellular or intracellular domain in screening for drugs that interact with HUMAN OCR10. Novel agents that bind to the receptor(s) described herein may mediate survival and differentiation in cells naturally expressing the receptor, but also may confer survival and proliferation when used to treat cells engineered to express the receptor. In particular embodiments, the extracellular domain (soluble receptor) of HUMAN OCR10 is utilized in screens for cognate ligands.

The invention also provides for a nucleic acid probe capable of hybridizing with a sequence included within the nucleic acid sequence encoding HUMAN OCR10 useful for the detection of HUMAN OCR10 expressing tissue in humans and animals.

The invention further provides for antibodies directed against HUMAN OCR10.

The present invention also has diagnostic and therapeutic utilities. In particular embodiments of the invention, methods of detecting aberrancies in the function or expression of the receptor described herein may be used in the diagnosis of endocrine or other disorders. In other embodiments, manipulation of the receptor or agonists which bind this receptor may be used in the treatment of, for example, endocrine disorders. In further embodiments, the extracellular domain of the receptor is utilized as a blocking agent which blocks the binding of ligand to target cells.

In a further embodiment of the invention, patients that suffer from an excess of HUMAN OCR10 may be treated by administering an effective amount of anti-sense RNA or anti-sense oligodeoxyribonucleotides corresponding to the HUMAN OCR10 gene coding region, thereby decreasing expression of HUMAN OCR10.

### DETAILED DESCRIPTION OF THE INVENTION

The invention provides HUMAN OCR10 polypeptides which include isolated HUMAN OCR10 polypeptides and recombinant polypeptides comprising a HUMAN OCR10 amino acid sequence, or a functional HUMAN OCR10 polypeptide domain thereof having an assay-discernable HUMAN OCR10-specific activity. Accordingly, the polypeptides may be deletion mutants of the disclosed HUMAN OCR10 polypeptide and may be provided as fusion products, e.g., with non-HUMAN OCR10 polypeptides. The subject HUMAN OCR10 polypeptides have HUMAN OCR10-specific activity or function.

A number of applications for HUMAN OCR10 polypeptides are suggested from their properties. HUMAN OCR10 polypeptides may be useful in the study and treatment of conditions similar to those which are treated using cytokines and/or hormones. Furthermore, the HUMAN OCR10 cDNA may be useful as a diagnostic tool, such as through the use of oligonucleotides as primers in a PCR test to amplify those sequences having similarities to the oligonucleotide primer, and to see how much HUMAN OCR10 mRNA is present in a particular tissue or sample. The isolation of HUMAN OCR10, of course, also provides the key to isolate its putative ligand, other HUMAN OCR10 binding polypeptides, and/or to study its properties.

HUMAN OCR10-specific activity or function may be determined by convenient in vitro, cell based or in vivo assays. In vitro or cell based assays include but are not limited to binding assays and cell culture assays. In vivo assays include but are not limited to immune response, gene therapy and transgenic animals. Binding assays encompass any assay where the specific molecular interaction of a HUMAN OCR10 polypeptide with a binding target is evaluated. The binding target may be a natural binding target, or a nonnatural binding target such as a specific immune polypeptide such as an antibody, or a HUMAN OCR10-specific binding agent.

The claimed HUMAN OCR10 polypeptides may be isolated or pure—an “isolated” polypeptide is one that is no longer accompanied by some of the material with which it is associated in its natural state, and that preferably constitutes at least about 0.5%, and more preferably at least about 5% by weight of the total polypeptide in a given sample; a “pure” polypeptide constitutes at least about 90%, and preferably at least about 99% by weight of the total polypeptide in a given sample. The subject polypeptides may be synthesized, produced by recombinant technology, or purified from cells. A wide variety of molecular and biochemical methods are available for biochemical synthesis, molecular expression and purification of the subject compositions, see e.g., Molecular Cloning, A Laboratory Manual (Sambrook, et al., Cold Spring Harbor Laboratory, Cold Spring Harbor, N.Y.), Current Protocols in Molecular Biology (Eds. Ausubel, et al., Greene Publ. Assoc., Wiley-lnterscience, N.Y.).

The subject polypeptides find a wide variety of uses including but not limited to use as immunogens, targets in screening assays, bioactive reagents for modulating cell growth, differentiation and/or function. For example, the invention provides methods for modifying the physiology of a cell comprising contacting the extracellular surface of the cell or medium surrounding the cell with an exogenous HUMAN OCR10 polypeptide under conditions whereby the added polypeptide specifically interacts with a component of the medium and/or the extracellular surface to effect a change in the physiology of the cell. According to these methods, the extracellular surface includes plasma membrane-associated molecules.

The term “exogenous HUMAN OCR10 polypeptide” refers to polypeptides not made by the cell or, if so, expressed at non-natural levels, times or physiologic locales. Media, include, but are not limited to, in vitro culture media and/or physiological fluids such as blood, synovial fluid and lymph. The polypeptides may be introduced, expressed, or repressed in specific populations of cells by any convenient way, including but not limited to, microinjection, promoter-specific expression of recombinant protein or targeted delivery of lipid vesicles.

The invention provides HUMAN OCR10-specific binding agents, methods of identifying and making such agents, and their use in diagnosis, therapy and pharmaceutical development. HUMAN OCR10-specific binding agents include HUMAN OCR10-specific antibodies (See, e.g., Harlow and Lane (1988) Antibodies, A Laboratory Manual, Cold Spring Harbor Laboratory, Cold Spring Harbor, N.Y.) and also includes other binding agents identified it with assays such as one-, two- and three-hybrid screens, and non-natural binding agents identified in screens of chemical libraries such as described below. Agents of particular interest modulate HUMAN OCR10 polypeptide function.

The invention further provides for the production of secreted polypeptides consisting of the entire extracellular domain of HUMAN OCR10 fused to the human immunoglobulin gamma-1 constant region (IgG1 constant) or the human immunoglobulin gamma-1 Fc region (IgG1 Fc). This fusion polypeptide is called a HUMAN OCR10 “receptorbody” (RB), and would be normally expected to exist as a dimer in solution based on formation of disulfide linkages between individual IgG1 constant region or IgG1 Fc region tails. HUMAN OCR10 RB encoding nucleic acids may be part of expression vectors and may be incorporated into recombinant host cells, e.g., for expression and screening, for transgenic animals, or for functional studies such as the efficacy of candidate drugs for diseases associated with HUMAN OCR10 polypeptide-mediated signal transduction. Expression systems are selected and/or tailored to effect HUMAN OCR10 RB polypeptide structural and functional variants through alternative post-translational processing.

The invention provides HUMAN OCR10 nucleic acids, which find a wide variety of applications, including but not limited to, use as translatable transcripts, hybridization probes, PCR primers, or diagnostic nucleic acids, as well as use in detecting the presence of HUMAN OCR10 genes and gene transcripts and in detecting or amplifying nucleic acids encoding additional HUMAN OCR10 homologs and structural analogs.

The subject nucleic acids are of synthetic/non-natural sequences and/or are isolated, i.e., no longer accompanied by some of the material with which it is associated in its natural state, preferably constituting at least about 0.5%, more preferably at least about 5% by weight of total nucleic acid present in a given fraction, and usually recombinant, meaning they comprise a non-natural sequence or a natural sequence joined to a nucleotide(s) other than that to which it is joined on a natural chromosome. Nucleic acids comprising the nucleotide sequence disclosed herein and fragments thereof, contain such sequence or fragment at a terminus, immediately flanked by a sequence other than that to which it is joined on a natural chromosome, or flanked by a native flanking region fewer than 10 kb, preferably fewer than 2 kb, which is immediately flanked by a sequence other than that to which it is joined on a natural chromosome. While the nucleic acids are usually RNA or DNA, it is often advantageous to use nucleic acids comprising other bases or nucleotide analogs to provide, for example, modified stability.

The sequence of the disclosed HUMAN OCR10 nucleic acid is used to obtain the deduced HUMAN OCR10 polypeptide sequence. Further, the sequence of the disclosed HUMAN OCR10 nucleic acid is optimized for selected expression systems (Holler, et al., (1993) Gene 136:323-328; Martin, et al., (1995) Gene 154:150-166) or used to generate degenerate oligonucleotide primers and probes for use in the isolation of natural HUMAN OCR10 encoding nucleic acid sequences (“GCG” software, Genetics Computer Group, Inc., Madison, Wis.). HUMAN OCR10 encoding nucleic acids may be part of expression vectors and may be incorporated into recombinant host cells, e.g., for expression and screening, for transgenic animals, or for functional studies such as the efficacy of candidate drugs for diseases associated with HUMAN OCR10 polypeptide-mediated signal transduction. Expression systems are selected and/or tailored to effect HUMAN OCR10 polypeptide structural and functional variants through alternative post-translational processing.

The invention also provides for nucleic acid hybridization probes and replication/amplification primers having a HUMAN OCR10 cDNA-specific sequence and sufficient to effect specific hybridization with SEQ. NO. 1. Demonstrating specific hybridization generally requires stringent conditions, for example, hybridizing in a buffer comprising 30% formamide in 5×SSPE (0.18 M NaCl, 0.01 M NaPO₄, pH 7.7, 0.001 M EDTA) buffer at a temperature of 42° C. and remaining bound when subject to washing at 42° C. with 0.2×SSPE; preferably hybridizing in a buffer comprising 50% formamide in 5×SSPE buffer at a temperature of 42° C. and remaining bound when subject to washing at 42° C. with 0.2×SSPE buffer at 42° C. HUMAN OCR10 cDNA homologs can also be distinguished from one another using alignment algorithms, such as BLASTX (Altschul, et al., (1990) Basic Local Alignment Search Tool, J. Mol. Biol. 215:403-410).

HUMAN OCR10 hybridization probes find use in identifying wild-type and mutant alleles in clinical and laboratory samples. Mutant alleles are used to generate allele-specific oligonucleotide (ASO) probes for high-throughput clinical diagnoses. HUMAN OCR10 nucleic acids are also used to modulate cellular expression or intracellular concentration or availability of active HUMAN OCR10 polypeptides. HUMAN OCR10 inhibitory nucleic acids are typically antisense—single stranded sequences comprising complements of the disclosed HUMAN OCR10 coding sequences. Antisense modulation of the expression of a given HUMAN OCR10 polypeptide may employ antisense nucleic acids operably linked to gene regulatory sequences. Cells are transfected with a vector comprising a HUMAN OCR10 sequence with a promoter sequence oriented such that transcription of the gene yields an antisense transcript capable of binding to endogenous HUMAN OCR10 encoding mRNA. Transcription of the antisense nucleic acid may be constitutive or inducible and the vector may provide for stable extrachromosomal maintenance or integration. Alternatively, single-stranded antisense nucleic acids that bind to genomic DNA or mRNA encoding a given HUMAN OCR10 polypeptide may be administered to the target cell, in or temporarily isolated from a host, at a concentration that results in a substantial reduction in expression of the targeted polypeptide. An enhancement in HUMAN OCR10 expression is effected by introducing into the targeted cell type HUMAN OCR10 nucleic acids which increase the functional expression of the corresponding gene products. Such nucleic acids may be HUMAN OCR10 expression vectors, vectors which upregulate the functional expression of an endogenous allele, or replacement vectors for targeted correction of mutant alleles. Techniques for introducing the nucleic acids into viable cells are known in the art and include, but are not limited to, retroviral-based transfection or viral coat protein-liposome mediated transfection.

The invention provides efficient methods of identifying agents, compounds or lead compounds for agents active at the level of HUMAN OCR10 modulatable cellular function. Generally, these screening methods involve assaying for compounds which modulate the interaction of HUMAN OCR10 with a natural HUMAN OCR10 binding target. A wide variety of assays for binding agents are provided including, but not limited to, protein-protein binding assays, immunoassays, or cell based assays. Preferred methods are amenable to automated, cost-effective, high throughput screening of chemical libraries for lead compounds.

In vitro binding assays employ a mixture of components including a HUMAN OCR10 polypeptide, which may be part of a fusion product with another peptide or polypeptide, e.g., a tag for detection or anchoring. The assay mixtures comprise a natural HUMAN OCR10 binding target. While native binding targets may be used, it is frequently preferred to use portions thereof as long as the portion provides binding affinity and avidity to the subject HUMAN OCR10 conveniently measurable in the assay. The assay mixture also comprises a candidate pharmacological agent. Candidate agents encompass numerous chemical classes, though typically they are organic compounds, preferably small organic compounds, and are obtained from a wide variety of sources including libraries of synthetic or natural compounds. A variety of other reagents such as salts, buffers, neutral proteins, e.g., albumin, detergents, protease inhibitors, nuclease inhibitors, or antimicrobial agents may also be included. The mixture components can be added in any order that provides for the requisite bindings and incubations may be performed at any temperature which facilitates optimal binding. The mixture is incubated under conditions whereby, but for the presence of the candidate pharmacological agent, the HUMAN OCR10 polypeptide specifically binds the binding target, portion or analog with a reference binding affinity. Incubation periods are chosen for optimal binding but are also minimized to facilitate rapid, high throughput screening.

After incubation, the agent-biased binding between the HUMAN OCR10 polypeptide and one or more binding targets is detected by any convenient way. For cell-free binding type assays, a separation step is often used to separate bound from unbound components. Separation may be effected by any number of methods that include, but are not limited to, precipitation or immobilization followed by washing by, e.g., membrane filtration or gel chromatography. For cell-free binding assays, one of the components usually comprises or is coupled to a label. The label may provide for direct detection as radioactivity, luminescence, optical or electron density, or indirect detection such as an epitope tag or an enzyme. A variety of methods may be used to detect the label depending on the nature of the label and other assay components, including but not limited to, through optical or electron density, radiative emissions, nonradiative energy transfers, or indirectly detected with, as a nonlimiting example, antibody conjugates. A difference in the binding affinity of the HUMAN OCR10 polypeptide to the target in the absence of the agent as compared with the binding affinity in the presence of the agent indicates that the agent modulates the binding of the HUMAN OCR10 polypeptide to the corresponding binding target. A difference, as used herein, is statistically significant and preferably represents at least a 50%, more preferably at least a 90% difference.

The invention provides for a method for modifying the physiology of a cell comprising an extracellular surface in contact with a medium, said method comprising the step of contacting said medium with an exogenous HUMAN OCR10 polypeptide under conditions whereby said polypeptide specifically interacts with at least one of the components of said medium to effect a change in the physiology of said cell.

The invention further provides for a method for screening for biologically active agents, said method comprising the steps of a) incubating a HUMAN OCR10 polypeptide in the presence of a HUMAN OCR10 polypeptide-specific binding target and a candidate agent, under conditions whereby, but for the presence of said agent, said polypeptide specifically binds said binding target at a reference affinity; b) detecting the binding affinity of said polypeptide to said binding target to determine an agent-biased affinity, wherein a difference between the agent-biased affinity and the reference affinity indicates that said agent modulates the binding of said polypeptide to said binding target.

One embodiment of the invention is an isolated HUMAN OCR10 polypeptide comprising the amino acid sequence as set forth herein or a fragment thereof having HUMAN OCR10-specific activity.

Another embodiment of the invention is a recombinant nucleic acid encoding HUMAN OCR10 polypeptide comprising the amino acid sequence as set forth herein or a fragment thereof having HUMAN OCR10-specific activity.

Still another embodiment is an isolated nucleic acid comprising a nucleotide sequence as set forth herein in SEQ. NO. 1 or a fragment thereof having at least 18 consecutive bases and which can specifically hybridize with a nucleic acid having the sequence of native HUMAN OCR10.

The present invention also provides for antibodies to the HUMAN OCR10 polypeptides described herein which are useful for detection of the polypeptides in, for example, diagnostic applications. For preparation of monoclonal antibodies directed toward HUMAN OCR10 polypeptides, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in “Monoclonal Antibodies and Cancer Therapy”, Alan R. Liss, Inc. pp. 77-96) and the like are within the scope of the present invention.

The monoclonal antibodies for diagnostic or therapeutic use may be human monoclonal antibodies or chimeric human-mouse (or other species) monoclonal antibodies. Human monoclonal antibodies may be made by any of numerous techniques known in the art (e.g., Teng et al., 1983, Proc. Natl. Acad. Sci. U.S.A. 80:7308-7312; Kozbor et al., 1983, Immunology Today 4:72-79; Olsson et al., 1982, Meth. Enzymol. 92:3-16). Chimeric antibody molecules may be prepared containing a mouse antigen-binding domain with human constant regions (Morrison et al., 1984, Proc. Natl. Acad. Sci. U.S.A. 81:6851, Takeda et al., 1985, Nature 314:452).

Various procedures known in the art may be used for the production of polyclonal antibodies to the HUMAN OCR10 polypeptides described herein. For the production of antibody, various host animals can be immunized by injection with the HUMAN OCR10 polypeptides, or fragments or derivatives thereof, including but not limited to rabbits, mice and rats. Various adjuvants may be used to increase the immunological response, depending on the host species, including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (Bacille Calmette-Guerin) and Corynebacterium parvum.

A molecular clone of an antibody to a selected HUMAN OCR10 polypeptide epitope can be prepared by known techniques. Recombinant DNA methodology (see e.g., Maniatis et al., 1982, Molecular Cloning, A Laboratory Manual, Cold Spring Harbor Laboratory, Cold Spring Harbor, N.Y.) may be used to construct nucleic acid sequences which encode a monoclonal antibody molecule, or antigen binding region thereof.

The present invention provides for antibody molecules as well as fragments of such antibody molecules. Antibody fragments which contain the idiotype of the molecule can be generated by known techniques. For example, such fragments include, but are not limited to, the F(ab′)₂ fragment which can be produced by pepsin digestion of the antibody molecule; the Fab′ fragments which can be generated by reducing the disulfide bridges of the F(ab′)₂ fragment, and the Fab fragments which can be generated by treating the antibody molecule with papain and a reducing agent. Antibody molecules may be purified by known techniques including, but not limited to, immunoabsorption or immunoaffinity chromatography, chromatographic methods such as HPLC. (high performance liquid chromatography), or a combination thereof.

The following example is offered by way of illustration and not by way of limitation.

### EXAMPLE 1

OCR10 was initially detected in tblastn searches of the non-redundant nucleotide database (NT) at The National Center for Biotechnology Information (NCBI), using sequences derived from members of the cytokine receptor family as queries. The matching region corresponded to a characteristic cytokine receptor family amino acid pattern WSXWS (Bazan, J. F., 1990, PNAS 87:6934-6938), located within a BAC. clone (Genebank Identification No. 2342739) derived from human chromosome 16. The nucleotide and deduced amino acid sequences of these regions correspond to nucleotides (NTs) 508-685 of SEQ. NO. 1 and amino acids 170-230 of forth below:

| | |
|---|---|
| | 10 20 30 40 50 60 |
| | * * * * * * * * * * * * |
| SEQ. NO. 1 | ATG CCG CGT GGC TGG GCC GCC CCC TTG CTC CTG CTG CTG CTC CAG GGA GGG TGG GGC TGC |
| SEQ. NO. 2 | M P R G W A A P L L L L L L Q G G W G C> |
| | 70 80 90 100 110 120 |
| | * * * * * * * * * * * * |
| | CCC GAC CTC GTC TGC TAC ACC GAT TAC CTC CAG ACG GTC ATC TGC ATC CTG GAA ATG TGG |
| | P D L V C Y T D Y L Q T V I C I L E M W> |
| | 130 140 150 160 170 180 |
| | * * * * * * * * * * * * |
| | AAC CTC CAC CCC AGC ACG CTC ACC CTT ACC TGG CAA GAC CAG TAT GAA GAG CTG AAG GAC |
| | N L H P S T L T L T W Q D Q Y E E L K D> |
| | 190 200 210 220 230 240 |
| | * * * * * * * * * * * * |
| | GAG GCC ACC TCC TGC AGC CTC CAC AGG TCG GCC CAC AAT GCC ACG CAT GCC ACC TAC ACC |
| | E A T S C S L H R S A H N A T H A T Y T> |
| | 250 260 270 280 290 300 |
| | * * * * * * * * * * * * |
| | TGC CAC ATG GAT GTA TTC CAC TTC ATG GCC GAC GAC ATT TTC AGT GTC AAC ATC ACA GAC |
| | C H M D V F H F M A D D I F S V N I T D> |
| | 310 320 330 340 350 360 |
| | * * * * * * * * * * * * |
| | CAG TCT GGC AAC TAC TCC CAG GAG TGT GGC AGC TTT CTC CTG GCT GAG AGC ATC AAG CCG |
| | Q S G N Y S Q E C G S F L L A E S I K P> |
| | 370 380 390 400 410 420 |
| | * * * * * * * * * * * * |
| | GCT CCC CCT TTC AAC GTG ACT GTG ACC TTC TCA GGA CAG TAT AAT ATC TCC TGG CGC TCA |
| | A P P F N V T V T F S G Q Y N I S W R S> |
| | 430 440 450 460 470 480 |
| | * * * * * * * * * * * * |
| | GAT TAC GAA GAC CCT GCC TTC TAC ATG CTG AAG GGC AAG CTT CAG TAT GAG CTG CAG TAC |
| | D Y E D P A F Y M L K G K L Q Y E L Q Y> |
| | 490 500 510 520 530 540 |
| | * * * * * * * * * * * * |
| | AGG AAC CGG GGA GAC CCC TGG GCT GTG AGT CCG AGG AGA AAG CTG ATC TCA GTG GAC TCA |
| | R N R G D P W A V S P R R K L I S V D S> |
| | 550 560 570 580 590 600 |
| | * * * * * * * * * * * * |
| | AGA AGT GTC TCC CTC CTC CCC CTG GAG TTC CGC AAA GAC TCG AGC TAT GAG CTG CAG GTG |
| | R S V S L L P L E F R K D S S Y E L Q V> |
| | 610 620 630 640 650 660 |
| | * * * * * * * * * * * * |
| SEQ. NO. 1 | CGG GCA GGG CCC ATG CCT GGC TCC TCC TAC CAG GGG ACC TGG AGT GAA TGG AGT GAC CCG |
| SEQ. NO. 2 | R A G P M P G S S Y Q G T W S E W S D P> |
| | 670 680 690 700 710 720 |
| | * * * * * * * * * * * * |
| | GTC ATC TTT CAG ACC CAG TCA GAG GAG TTA AAG GAA GGC TGG AAC CCT CAC CTG CTG CTT |
| | V I F Q T Q S E E L K E G W N P H L L L> |
| | 730 740 750 760 770 780 |
| | * * * * * * * * * * * * |
| | CTC CTC CTG CTT GTC ATA GTC TTC ATT CCT GCC TTC TGG AGC CTG AAG ACC CAT CCA TTG |
| | L L L L V I V F I P A F W S L K T H P L> |
| | 790 800 810 820 830 840 |
| | * * * * * * * * * * * * |
| | TGG AGG CTA TGG AAG AAG ATA TGG GCC GTC CCC AGC CCT GAG CGG TTC TTC ATG CCC CTG |
| | W R L W K K I W A V P S P E R F F M P L> |
| | 850 860 870 |
| | * * * * * * |
| | TAC AAG GGC TGC AGC GGA GAC TTC AAG TGA |
| | Y K G C S G D F K *> |

The HMMR program (http://hmmer.wustl.edu) was used to find another region matching a cytokine receptor family amino acid pattern on the same BAC sequence. This region corresponds to a proline hinge motif PP, which is normally adjacent to the WSXWS region. The nucleotide and deduced amino acid sequences of the proline hinge motif region correspond to NTs 352-507 of SEQ. NO. 1 and amino acids 118-169 of SEQ. NO. 2, respectively. These data suggested that two exons of a novel cytokine receptor have been located on the BAC clone.

### EXAMPLE 2

### Cloning of human OCR10 gene using PCR.

A 111-mer oligonucleotide was synthesized (Genelink, Thornwood, N.Y.) that corresponded to NTs 568-678 of SEQ. NO. 1 for use as a PCR template. Two smaller oligonucleotides, hOCR10-1 (NTs 568-585 of SEQ. NO. 1) and hOCR10-2rc (NTs 660-678 of SEQ. NO. 1), corresponding to the outer regions of the OCR10 111-mer oligonucleotide, were also synthesized (Genelink, Thornwood, N.Y.) and used as amplification primers using the 111-mer oligonucleotide as a template in a standard PCR reaction. The resulting PCR product was used to probe a Northern blot (CLONTECH Human Multiple Tissue Blot, Catalog #7760-1) at an overnight hybridization temperature of 65° C., a wash temperature of 65° C., and an Bio-Imaging Analyzer BAS 2000 (Fugi) exposure time of 22 hours. Faint 9.5 kb RNA transcripts were observed in two human tissues, heart and placenta.

The WSXWS and proline hinge motif region sequences were pieced together theoretically (resulting sequence corresponding to NTs 352-685 of SEQ. NO. 1) and several oligonucleotides were synthesized (Genelink, Thornwood, N.Y.) that corresponded to specific sequences within each of these regions. These oligonucleotides were used in the following PCR reactions using standard PCR reaction conditions.

PCR reaction #1 was carried out with oligonucleotides hOCR10.5 (NTs 372-395 of SEQ. NO. 1) and hOCR10.2rc (NTs 660-678 of SEQ. NO. 1) using CLONTECH's Marathon-Ready™ cDNA derived from eight different tissues (Human Pancreas, catalog #7410-1; Human Heart, catalog #7404-1; Human Fetal Liver, catalog #7403-1; Human Fetal Skeletal Muscle, catalog #7435-1; Human Fetal Spleen, catalog #7422-1; Human Spleen, catalog #7412-1; Human Fetal Brain, catalog #7402-1; and Human Lung, catalog #7408-1) as PCR templates. None of the reactions produced visible PCR products when run on a 1% agarose gel.

PCR reaction #2, a nested PCR reaction, was carried out with oligonucleotides hOCR10.6 (NTs 414-437 of SEQ. NO. 1) and hOCR10.4rc (NTs 635-658 of SEQ. NO. 1) using as the PCR templates the products of the eight PCR reactions from PCR reaction #1 supra. The strongest 240 bp PCR fragment was obtained in human fetal spleen, human spleen, and human lung. The other tissues tested, human pancreas, heart, fetal liver, fetal skeletal muscle, and fetal brain, produced only faint 240 bp PCR products.

The 240 bp PCR fragment was sequenced by standard techniques using an ABI 373A DNA sequencer and Taq Dideoxy Terminator Cycle Sequencing Kit (Applied Biosystems, Inc., Foster City, Calif.) and found to contain DNA sequence homologous to both the WSXWS and the proline hinge motif regions. The nucleotide and deduced amino acid sequences of the 240 bp PCR fragment corresponds to NTs 418-651 of SEQ. NO. 1 and amino acids 140-217 of SEQ. NO. 2, respectively.

### EXAMPLE 3

### 5′RACE to obtain complete 5′ region of HUMAN OCR10.

A 5′ RACE (CLONTECH Marathon-Ready™ cDNA user manual #PT1156-1) was performed on human spleen and human lung cDNA (CLONTECH's Marathon-Ready™ cDNA, catalog #7412-1 and #7408-1, respectively) in an attempt to clone additional 5′ sequence.

The first PCR reaction was performed with the 5′ oligonucleotide hOCR10.2rc (NTs 660-678 of SEQ. NO. 1) and the RACE kit oligonucleotide AP1. This amplification produced no visible PCR product.

The second PCR reaction was performed with the 5′ oligonucleotide hOCR10.4rc (NTs 635-658 of SEQ. NO. 1) and the RACE kit oligonucleotide AP2. PCR product smears were obtained from this reaction. Five microliters of each reaction was run on a 1% agarose gel, the gel was denatured and neutralized by standard techniques (See Current Protocols in Molecular Biology, Eds. Ausubel, et al., Greene Publ. Assoc., Wiley-lnterscience, N.Y.), dried on a Savant Slab Gel Dryer (Savant, Holbrook, N.Y.) and hybridized overnight at 45° C. using a nested oligonucleotide probe (hOCR10.7 nt 441-465). Both of the 5′-specific RACE reaction smears ranged in size from about 200 bp to 2500 bp. A preparative 1% agarose gel was run and six individual slices were cut from varying regions of the smears and purified using QIAEX II Gel Extraction Kit, (catalog #20021, QIAGEN, Valencia, Calif.). The purified slices were subcloned into Zeroblunt (catalog #K2700-20, Invitrogen, Carlsbad, Calif.). The new 5′ sequence (NTs 1-352 of SEQ. NO. 1) was confirmed using an ABI 373A DNA sequencer and Taq Dideoxy Terminator Cycle Sequencing Kit (Applied Biosystems, Inc., Foster City, Calif.). The newly obtained sequence revealed the presence of a MET start codon (amino acid number 1 of SEQ. NO. 2), a secretion signal (amino acid numbers 4-21 of SEQ. NO. 2), and the remaining portion of the ligand binding domain including a characteristic cytokine receptor family cysteine pattern (amino acids 25, 35, 65, and 81 of SEQ. NO. 2) located within this region.

### EXAMPLE 4

### 3′RACE to obtain complete 3′ region of HUMAN OCR10

A 3′ RACE reaction was performed using human spleen and human lung Marathon-Ready™ cDNA from CLONTECH as follows:

The first PCR reaction was carried out with oligonucleotide hOCR10.5 (NTs 372-395 of SEQ. NO. 1) and the RACE kit oligonucleotide AP1. This reaction produced no visible PCR product.

The second PCR reaction was carried out with oligonucleotide hOCR10.6 (NTs 414-437 of SEQ. NO. 1) and RACE kit oligonucleotide AP2. This reaction produced multiple bands plus smears. Five microliters of each reaction was loaded on a 1% agarose gel, the gel was denatured and neutralized by standard techniques (See Current Protocols in Molecular Biology, Eds. Ausubel, et al., Greene Publ. Assoc., Wiley-Interscience, N.Y.), dried on a Savant Slab Gel Dryer and hybridized overnight at 45° C. with a nested oligonucleotide hOCR10.7 (NTs 441-465 of SEQ. NO. 1). Both of the 3′-specific RACE reaction smears hybridized to a band of approximately 2500 bp. A preparative 1% agarose gel was run and 7 individual slices were cut from varying regions of the smears, including the region corresponding to the 2500 bp band that hybridized specifically. The individual slices were purified using QIAEX II Gel Extraction Kit (catalog #20021, QIAGEN, Valencia, Calif.) and each of these individual slices plus the original second PCR reaction (not gel-purified) were amplified for a third time with another nested oligonucleotide, hOCR10.7 (NTs 441-465 of SEQ. NO. 1), and RACE kit oligonucleotide AP2. Again, multiple bands were obtained. Five microliters of each reaction was loaded on a 1% agarose gel, the gel was denatured, neutralized and dried and hybridized overnight at 45° C. with nested oligonucleotide hOCR10.9 (NTs 469-490 of SEQ. NO. 1). This hybridization revealed a specific band of approximately 750 bp. A preparative 1% agarose gel was run and this 750 bp band was isolated and purified using QIAEX II Gel Extraction Kit. The purified slices were sequenced by standard techniques and the 3′ end sequence, corresponding to NTs 686-870 of SEQ. NO. 1, contained a stop codon.

HUMAN OCR10 appears to be a typical member of the cytokine receptor family. It has sequences resembling a secretion signal (amino acids 4-21 of SEQ. NO. 2); a typical cytokine receptor family ligand-binding domain comprised of approximately 200 amino acids that contains in its extracellular domain 4 conserved cysteines (amino acids 25, 35, 65, and 81 of SEQ. NO. 2), a proline hinge motif (PP) at amino acids 122-123 of SEQ. NO. 2, and a characteristic cytokine receptor family WSXWS amino acid pattern (amino acids 214-218 of SEQ. NO. 2). In addition, there is a. putative hydrophobic transmembrane domain comprising amino acids 238-255 of SEQ. NO. 2; and a potential Jak-binding region (See Cohen, et al., 1995, Cell 80:237-248) at amino acids 263-278 of SEQ. NO. 2. HUMAN OCR10's most closely related cytokine receptor family members includes, but is not limited to, IL-9 receptor (Genebank Identification No. 632993), the cytokine receptor common P chain (Genebank Identification No. 416868), the EPO receptor (Genebank Identification No. 119524), and leptin receptor (Genebank Identification No. 2760950). All sequences identified above by Genebank Identification Nos. can be obtained from The National Center for Biotechnology Information (NCBI) database by accessing their website address www.ncbi.nlm.nih.gov/entrez/protein.html.

Thus, HUMAN OCR10 appears to be a receptor for a known or novel cytokine. It may be used either to identify and clone the novel cytokine, or to control the signaling by the known one.

The present invention is not to be limited in scope by the specific embodiments described herein. Indeed, various modifications of the invention in addition to those described herein will become apparent to those skilled in the art from the foregoing description and accompanying figures. Such modifications are intended to fall within the scope of the appended claims.

What is claimed is:

1. An isolated nucleic acid molecule encoding HUMAN OCR10.

2. An isolated nucleic acid molecule according to claim 1, having a sequence selected from the group consisting of:
(a) the nucleotide sequence comprising the coding region of the HUMAN OCR10 as set forth in SEQ. NO. 1;
(b) a nucleotide sequence that hybridizes under stringent conditions to the nucleotide sequence of (a) and which encodes a molecule having the activity of the HUMAN OCR10; or
(c) a nucleotide sequence which, but for the degeneracy of the genetic code would hybridize to a nucleotide sequence of (a) or (b), and which encodes a molecule having the activity of the HUMAN OCR10.

3. A vector which comprises a nucleic acid molecule of claim 1.

4. A vector according to claim 3, wherein the nucleic acid molecule is operatively linked to an expression control sequence capable of directing its expression in a host cell.

5. An isolated nucleic acid molecule encoding a HUMAN OCR10.

6. Isolated HUMAN OCR10 polypeptide.

7. Isolated HUMAN OCR10 polypeptide encoded by the nucleic acid molecule of claim 2.

8. A host-vector system for the production of HUMAN OCR10 polypeptide which comprises a vector of claim 4, in a host cell.

9. A host-vector system according to claim 8, wherein the host cell is a bacterial, yeast, insect or mammalian cell.

10. A method of producing HUMAN OCR10 polypeptide which comprises growing cells of a host-vector system of claim 9, under conditions permitting the production of HUMAN OCR10 polypeptide and recovering the HUMAN OCR10 polypeptide so produced.

11. An antibody which specifically binds HUMAN OCR10 polypeptide of claim 6.

12. An antibody according to claim 11, which is a monoclonal antibody.

13. A composition comprising HUMAN OCR10 polypeptide according to claim 7 and a carrier.

14. A composition comprising an antibody according to claim 11, and a carrier.

15. A composition comprising the extracellular portion of the HUMAN OCR10 polypeptide fused to an immunoglobulin constant region.

16. The composition of claim 15, wherein the constant region is the human immunoglobulin gamma-1 constant region.

17. A composition comprising the extracellular portion of the HUMAN OCR10 polypeptide fused to an immunoglobulin Fc region.

18. The composition of claim 17, wherein the Fc region is the human immunoglobulin gamma-1 Fc region.
